(19) United States
(12) Patent Application Publication
(10) Pub. No.: US 2025/0275783 A1
(43) Pub. Date: Sep. 4, 2025

(54) Percutaneous Cardiac Tissue Cutter

(71) Applicant: Michael B. McDonald, Cordova, TN (US)

(72) Inventor: Michael B. McDonald, Cordova, TN (US)

(21) Appl. No.: 19/211,676

(22) Filed: May 19, 2025

Related U.S. Application Data
(63) Continuation-in-part of application No. 18/511,054, filed on Nov. 16, 2023.
(60) Provisional application No. 63/648,960, filed on May 17, 2024.
(60) Provisional application No. 63/425,708, filed on Nov. 16, 2022.

(51) Int. Cl. A61B 17/32; A61B 17/00

(52) U.S. Cl. CPC A61B 17/32; A61B 17/00234; A61B 2017/00243; A61B 2017/00305; A61B 2017/00367; A61B 2017/00862

(57) ABSTRACT

A cardiac cutter for cutting cardiac valve leaflets provides a main catheter adapted for femoral insertion into a patient's vasculature in an over-the-wire method. The main catheter is deployable within a patient's heart and has a proximal bridge tube and a distal bridge tube with a cutting wire extending between them, the proximal bridge tube, distal bridge tube, and cutting wire together forming a bridge that extends outwardly from the main catheter. The cutting wire is configured to cut cardiac valve leaflets using a reciprocal motion or rotating motion, and is operable by a controller. The cutting wire is threaded through a foot positioned between the proximal bridge tube and the distal bridge tube. The foot aids in the positioning of the cutting wire against a leaflet to be cut. A commissure guide tube extends from the main catheter opposite from the cutting wire and serves to stabilize the cutter within the valve.

### REFERENCE TO RELATED APPLICATIONS

This application claims priority to U.S. Provisional Patent Application Ser. No. 63/648,960, entitled Percutaneous Mitral Valve Saw” and filed on May 17, 2024, which is fully incorporated herein by reference. This application is also a continuation-in-part of, and claims priority to, U.S. Non-Provisional application Ser. No. 18/511,054, titled “Percutaneous Cardiac Valve Cutter,” filed Nov. 16, 2023, which claimed priority to U.S. Provisional Patent Application Ser. No. 63/425,708, titled “Percutaneous Cardiac Valve Saw,” filed on Nov. 16, 2022. All of the foregoing are incorporated herein by reference in their entireties.

### BACKGROUND AND SUMMARY

In the emerging field of structural cardiology, there is often the need for cardiac leaflet modification. A cardiac leaflet modification may be done on a previously placed bioprosthetic valve or the native valve. Making cuts in the valve leaflets can mitigate problems created by the leaflets of the first valve. When placing a second valve inside the first valve, the leaflets of the first valve will be pinned up against the valve frames creating a tube of tissue between the two valve frames. The tissue tube formed by the first valve's leaflets is often referred to as the neoskirt. Making cuts in the leaflets of the original valve with a cutter according to the present disclosure will help prevent creating an intact tube of tissue between the two valves. An intact neoskirt can limit blood flow to the coronary arteries and limit coronary catheter access to the arteries.

A cardiac cutter according to the present disclosure is provided for cutting cardiac valve leaflets via a main catheter adapted for femoral insertion into a patient's vasculature in an over-the-wire method. The main catheter is deployable within a patient's heart and has a proximal bridge tube and a distal bridge tube with a cutting wire extending between them, the proximal bridge tube, distal bridge tube, and cutting wire together forming a bridge that extends outwardly from the main catheter. The cutting wire is configured to cut cardiac valve leaflets using a reciprocal motion or rotating motion and is operable by a controller. The cutting wire is threaded through a foot positioned between the proximal bridge tube and the distal bridge tube. The foot aids in the positioning of the cutting wire against a leaflet to be cut and prevents the cutting wire from cutting tissue not within the boundaries of an inner loop of the foot. A commissure guide tube extends from the main catheter opposite from the cutting wire and serves to stabilize the cutter within the valve.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein.

### DESCRIPTION OF THE DRAWINGS

FIG. 1 depicts a cutter according to an embodiment of the present disclosure.

FIG. 2 depicts a side plan view of the cutter of FIG. 1.

FIG. 3 depicts the cutter of FIG. 1 that has been advanced into a patient's aortic valve and deployed.

FIG. 4 depicts the cutter of FIG. 3, where the cutting wire has begun to cut into a leaflet of a patient's aortic valve.

FIG. 5 depicts the cutter of FIG. 3, where the cutting wire has completed cutting into a leaflet of a patient's aortic valve.

FIG. 6 is a cross-sectional view of the cutter of FIG. 3 within a patient's heart, taken along section lines A-A of FIG. 3.

FIG. 7 is a cross-sectional view of the cutter of FIG. 4 within a patient's heart, taken along section lines B-B of FIG. 4.

FIG. 8 is a cross-sectional view of the cutter of FIG. 5 within a patient's heart, taken along section lines C-C of FIG. 5.

FIG. 9 depicts a controller to an embodiment of the present disclosure.

FIG. 10 illustrates the main catheter of the cutter after it has been positioned in a patient's heart for cutting the leaflets of a bioprosthetic valve.

FIG. 11 depicts the cutter after it has been advanced into position in the bioprosthetic valve of FIG. 10 and the sheath has been retracted.

FIG. 12 depicts the cutter of FIG. 11 after the commissure guidewire has been deployed.

FIG. 13 depicts the cutter of FIG. 12 after the bridge has been partially deployed and

the leaflet partially cut.

FIG. 14 depicts the cutter of FIG. 13 after the bridge has been fully deployed and the leaflet fully cut.

FIG. 15 depicts the cutter of FIG. 14 after the bridge and commissure guidewire are undeployed following the cutting of the leaflet.

FIG. 16 depicts the cutter of FIG. 15 after the cutter has been partially withdrawn from the valve to reposition the cutter.

FIG. 17 depicts an alternative embodiment of a bridge of a cutter according to the present disclosure.

FIG. 18 depicts an enlarged view of the distal bridge tube and the distal bridge support, taken along detail line D of FIG. 17.

FIG. 19 depicts an alternative embodiment of a mitral valve version of a cutter according to the present disclosure.

FIG. 20 depicts an end view of the cutter of FIG. 19.

FIG. 21 depicts the cutter of FIG. 19 in an undeployed configuration.

### DETAILED DESCRIPTION

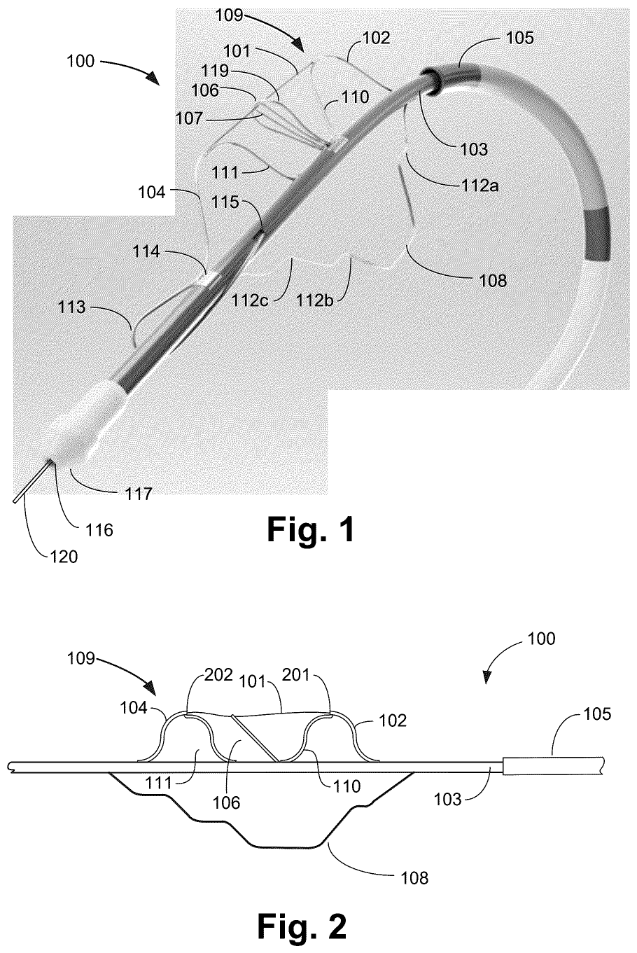

FIG. 1 is a perspective view of a cutter 100 according to an exemplary embodiment of the present disclosure, shown in a deployed configuration. In this embodiment, the cutter 100 comprises a main catheter 103 that houses a central wire 120 for directing the cutter 100 through a patient's blood vessels (not shown) and across a cardiac valve (not shown) that is to be cut. The main catheter 103 is adapted for femoral insertion into a patient's vasculature in an over-the-wire method.

The main catheter 103 houses a cutting wire 101 that extends outwardly from the main catheter 103 when deployed. In this regard, the cutting wire 101 extends between a proximal bridge tube 102 and a distal bridge tube 104. The proximal bridge tube 102 and the distal bridge tube 104 are both formed from hollow tubes (formed from nitinol in one embodiment), and the cutting wire 101 is threaded through the proximal bridge tube 102 and the distal bridge tube 104 such that the cutting wire 101 extends between them. The proximal bridge tube 102 and the distal bridge tube 104 project the cutting wire 101 out from the body of the main catheter 103 for the cutting operation. In the deployed configuration, the cutting wire 101 extends generally parallel to the shaft of the main catheter 103.

A proximal bridge support 110 is affixed to and supports the proximal bridge tube 102 when the cutter 100 is in a deployed configuration. A distal bridge support 111 is affixed to and supports the distal bridge tube 104 when the cutter is in the deployed configuration. In this regard, the proximal bridge support 110 and the distal bridge support 111 each extend from the main catheter 103 from their lower ends when the cutter 100 is in the deployed configuration, and their upper ends are affixed to and support the proximal bridge tube 102 and distal bridge tube 104, respectively. In this manner the proximal bridge support 110 and distal bridge support 111 assist in deploying the cutting wire 101 such that the cutting wire 101, the proximal bridge tube 102 and distal bridge tube 104 form a bridge 109 extending from the main catheter 103. The proximal bridge support 110 and the distal bridge support 111 are formed from flexible nitinol tubing in one embodiment.

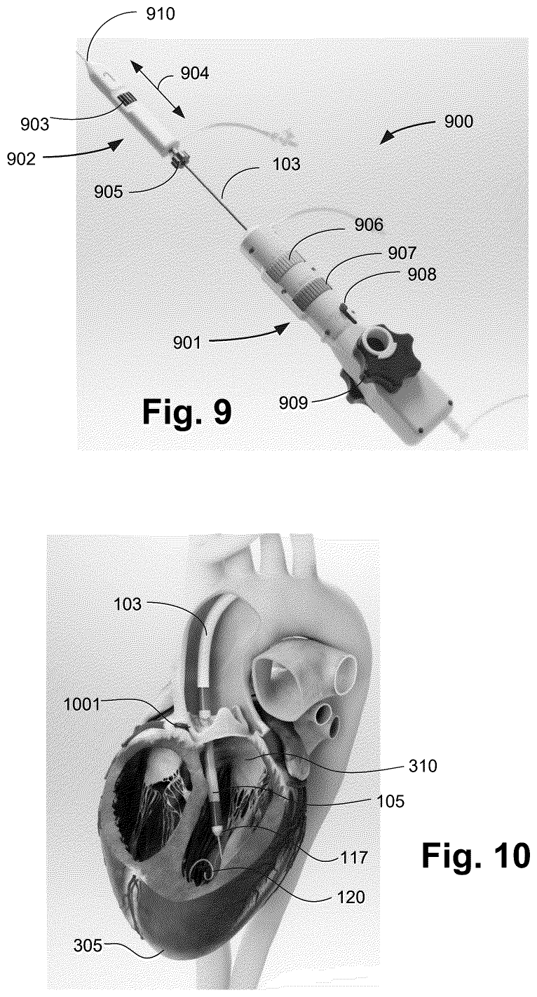

The cutting wire 101 further passes through the main catheter 103 to a controller (not shown) in the illustrated embodiment. Operation by the user of the controller controls the motion and tension of the cutting wire 101, as discussed herein with respect to FIG. 9.

A foot 106 extends from the main catheter 103 when the cutter 100 is in the deployed configuration. The foot 106 comprises an inner loop 107 and an outer loop 119 in the illustrated embodiment. The cutting wire 101 is threaded through the inner loop 107 of the foot 106, such that the foot 106 constrains the cutting wire 101 within the inner loop 107, while allowing the cutting wire 101 to freely move back and forth in a reciprocal fashion, or to rotate, while also preventing the cutting wire 101 from cutting into tissue that is not within the inner loop 107. The inner loop 107 is approximately 0.1 cm wide in one embodiment. The foot 106 helps to direct the cutting wire 101 during the cutting operation, as further discussed herein. The outer loop 119 surrounds the inner loop 107, adding more surface area to the foot and helping to center the foot on a leaflet (not shown) and stabilize the leaflet during the cutting operation. The outer loop 119 and the inner loop 107 are formed from flexible nitinol tubing in one embodiment.

A commissure guidewire 108 extends from the main catheter 103 when the commissure guidewire 108 is in the deployed configuration. The commissure guidewire 108 is disposed on an opposite side from (i.e., 180 degrees around the main catheter 103 from) the cutting wire 101. The commissure guidewire 108 serves to help position the cutting wire 101 during the cutting operation, as further discussed herein. The commissure guidewire 108 is formed from flexible nitinol wire in one embodiment, and comprises irregular steps 112a, 112b, and 112c in the illustrated embodiment. The steps 112a, 112b, and 112c are bends in the commissure guidewire 108 that may aid in stabilizing the cutting wire 101 during the cutting operation.

The central catheter 103 comprises an outer sheath 105 that extends to enclose the cutting wire 101, proximal bridge tube 102, distal bridge tube 104, foot 106, the proximal bridge support 110, distal bridge support 111, and commissure guidewire 108 when the catheter is being passed into a patient's body to its heart through vasculature. In this regard, the sheath 105 extends to contact a nosecone 117 of the cutter 100 when the cutter 100 is in an undeployed configuration. The outer sheath 105 is retractable to reveal the cutting wire 101 and other deployable components when the catheter 103 has been placed in position in the patient's heart, as further discussed herein. The nosecone 117 comprises a nosecone opening 116, the nosecone opening receiving the central wire 120 that the catheter 103 passes over.

As discussed herein, the proximal bridge tube 102, the distal bridge tube 104, the proximal bridge support 110, and distal bridge support 111 are formed from nitinol tubing in one embodiment. The nitinol tubing is flexible and straightens when undeployed and deploys to preset shapes as shown in the illustrated configuration when the bridge 109 is deployed.

In one embodiment, the foot 106 is formed from solid nitinol wire that is flexible and straightens when constrained by the sheath 105 and deploys to the preset shape as shown in the illustrated configuration when the sheath 105 is retracted.

The cutting wire 101 comprises a diamond cutting wire in one embodiment. The motion of the cutting wire 101 may be in a one-way fashion (rotating) or two-way fashion (back and forth in a sawing motion). In one embodiment, the ends (not shown) of the cutting wire 101 feed back through the main catheter 103 to a control handle of the controller, as further discussed below. In the illustrated embodiment, the distal bridge tube 104 is affixed to the main catheter 103 at its lower end at a connection point 114. The tubing that comprises the distal bridge tube 104 then is redirected at a turnaround point 114 and fed back into the main catheter at an opening 115 for feeding the tubing containing the cutting wire 101 back to the controller (not shown). Other embodiments may use other configurations to connect the distal bridge tube 104 to the main catheter 103 and other ways to return the tubing containing the cutting wire 101 back to the controller.

FIG. 2 is a side plan view of the cutter 100 of FIG. 1. The proximal bridge tube 102 has an opening (not shown) in its upper end 201 through which the cutting wire 101 exits, and the distal bridge tube 104 has an opening (not shown) in its upper end 202 through which the cutting wire 101 enters. The open end 201 of the proximal bridge tube 102 faces the open end 202 of the distal bridge tube 104 when the cutter 100 is in the deployed position, such that the cutting wire extends between and moves within the proximal bridge tube 102 and the distal bridge tube 104.

When the main catheter 103 is in a deployed configuration, the proximal bridge tube 102 extends from the main catheter 103 outwardly in a radial direction and distally towards the distal end of the cutter 100. The distal bridge tube 104 extends from the main catheter 103 outwardly in a radial direction and forwardly towards the proximal end of the cutter 100. The proximal bridge tube 102 and distal bridge tube 104 are in the same radial plane as one another, and both are in the same general plane a center of the foot 106.

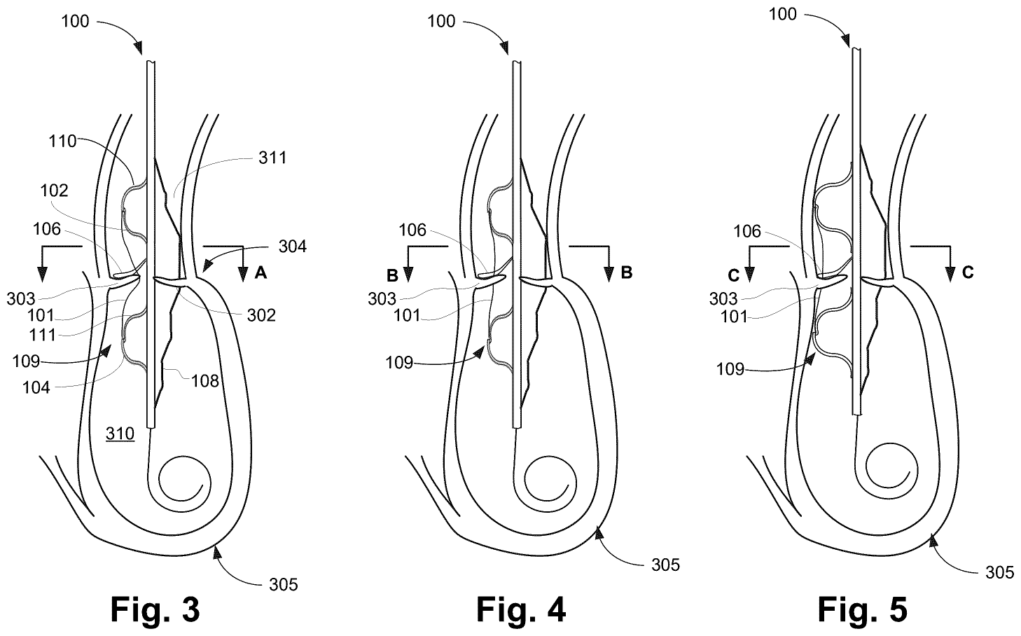

FIG. 3 is a cross-sectional depiction of a patient's heart 305, where the cutter 100 of FIG. 1 has been advanced into the aortic valve 304 and deployed to cut a leaflet 303 of a stenosed aortic valve. To use the cutter 100, the cutter 100 is advanced transfemorally through the patient's aorta 311 and across the aortic valve 304, such that after the sheath (not shown) is retracted and the bridge 109 is partially deployed, the distal bridge tube 104 and distal bridge support 111 have entered the left ventricle cavity 310. The cutter 100 is positioned such that the foot 106 is adjacent to or contacts the leaflet 303 desired to be cut, and the proximal bridge tube 102 is on the aortic side of the valve 304. In this orientation, the cutting wire 101 extends from the aorta 311, through the aortic valve 304, and into the left ventricular cavity 310. (Note that the bridge 109 in this illustration has been partially, but not fully deployed. FIG. 5 depicts the bridge 109 in a fully-deployed configuration.)

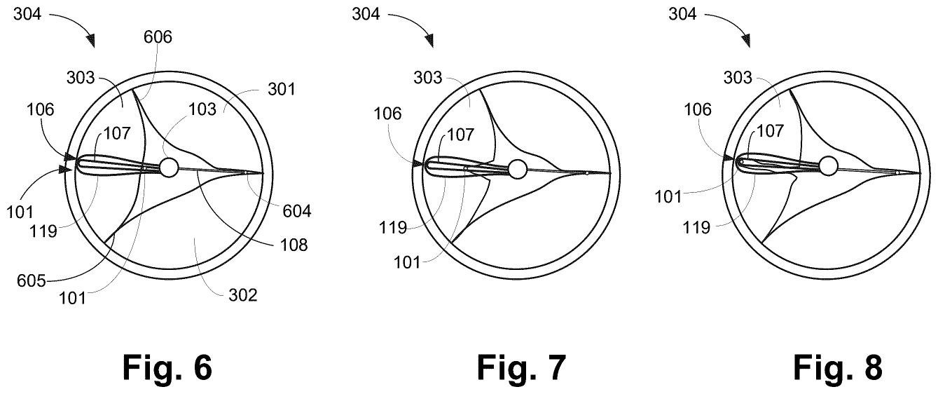

The foot 106 may contact, or be adjacent to, the inner wall of the aortic valve 304. The commissure guidewire 108 may rest in the commissure (not shown) separating the leaflets adjacent to leaflet 303, as further depicted in FIG. 6. (One such adjacent leaflet 302 is depicted in FIG. 3; see FIG. 6 for a top view of the valve 304 depicting all three leaflets of a valve.) The commissure guidewire 108 serves to stabilize the cutter 100 and helps to center the cutter 100.

As illustrated in FIG. 3, the cutting wire 101 is within the inner loop 107 of the foot 106. In this position with little tension applied to the cutting wire 101, and before the cutting wire motion has begun, the wire does not begin cutting into the leaflet.

FIG. 4 depicts the cutter 100 of FIG. 3, where tension has been applied to the cutting wire 101, and the cutting wire 101 has been moved up and down in a reciprocating motion, such that the cutting wire 101 has begun to slice into the leaflet 303. In this view the leaflet 303 has been cut about halfway through. In this embodiment, a reciprocating sawing motion of the cutting wire 101 has been used to cause the cutting wire 101 to cut into the leaflet 303. As discussed herein, other embodiments may employ a rotating cutting wire.

FIG. 5 depicts the cutter of FIG. 3, where the bridge 109 has been fully deployed and the cutting wire 101 has fully cut the leaflet 303. When the bridge was 109 only partially deployed, as illustrated in FIGS. 3 and 4, the cutting wire 101 could not cut fully through the leaflet 303. But when the bridge 109 is fully deployed as shown, the cutting wire 101 can slice through the leaflet 303, close to the wall of the valve but constrained within the foot 106 such that it cannot cut too far.

FIG. 6 is a cross-sectional view of the cutter 100 of FIG. 3, with the cutter 100 in a patient's aortic valve 304 as depicted in FIG. 3, taken along section lines A-A of FIG. 3. The valve 304 comprises leaflets 301, 302, and 303, and commissures 604, 605, and 606. In this illustration the foot 106 is pressing against or adjacent to the leaflet 303. The distal tip of the foot 106 is adjacent to the inner wall of the aortic valve 304. The cutting wire 101 is threaded through and retained within the inner loop 107 of the foot 106. The foot 106 thus guides the cutting wire 101 to cut only within the perimeter of the inner loop 107 of the foot 106.

The main catheter 103 is generally centered within the valve 304. The commissure guidewire 108 has been deployed to rest in the commissure 604 between leaflets 301 and 302. The commissure guidewire pressing against the commissure and/or against the inner wall of the aortic calve 304 helps to keep the main catheter 103 centered and to stabilize the cutter 100 during the cutting operation.

FIG. 7 is a cross-sectional view of the cutter 100 of FIG. 4, taken along section lines B-B of FIG. 4. In this view, the cutting wire 101 has cut almost halfway through the leaflet 303.

FIG. 8 is a cross-sectional view of the cutter 100 of FIG. 5, taken along section lines C-C of FIG. 5. In this view, the cutting wire 101 has cut all of the way through the leaflet 303, and has been stopped from cutting further by the inner loop 105 of the foot 106. After one leaflet has been cut, the cutter 100 can be repositioned to cut the remaining leaflets of the valve.

After all of the leaflets have been cut, the arm tube 104 and distal tube 102 are pulled back inside of the main catheter 103 (FIG. 1) and the sheath 105 (FIG. 1) is advanced to return the catheter to an undeployed configuration, and the main catheter 103 may then be removed from the patient's body.

FIG. 9 depicts a controller 900 used to control the cutter 100 (FIG. 1) according to an embodiment of the present disclosure. The controller 900 comprises a steerable control guide 902 and a main controller 901 in the illustrated embodiment. The main catheter 103 exits a distal end 910 of the steerable control guide 902. Moving the steerable control guide 902 with respect to the main controller 901 in the direction indicated by directional arrow 904 retracts and extends the sheath 105 (FIG. 1). In this regard, moving the steerable control guide 902 towards the main controller 901 retracts the sheath 105, and moving the steerable control guide 902 away from the main controller 901 extends the sheath. As illustrated in FIG. 9, the steerable control guide 902 is positioned such that the sheath is retracted.

The steerable control guide 902 comprises a steerable control actuator 903. The steerable control actuator 903 is actuated to move the distal end of the main catheter 103 (FIG. 1) to aid in positioning the cutter, via flexion and extension of the main catheter 103.

A touhy borst valve actuator 905 is positioned on the main catheter 103 between the steerable control guide 902 and the main controller 901. The touhy borst valve serves to prevent blood from leaking from around the catheter during the cutting procedure. The touhy borst valve actuator 905 is loosened before the sheath 105 is advanced or retracted, and tightened after the sheath 105 has been advanced or retracted to lock the sheath 105 in place.

The main controller 901 comprises a commissure guide control actuator 906 that when actuated, causes the commissure guidewire 108 (FIG. 1) to deploy. In the illustrated embodiment, the commissure guide control actuator 906 is a finger-operated dial that deploys the commissure guidewire 108 more the further the dial is turned. The user can dial the commissure guide control actuator 906 partially to partially deploy the commissure guidewire 108 and can dial the actuator 906 fully to fully deploy the commissure guidewire 108.

The main controller 901 further comprises a bridge control actuator 907 that when actuated, causes the bridge 109 (FIG. 1) to deploy. In the illustrated embodiment, the bridge control actuator 907 is a finger-operated dial that deploys the bridge 109 more the further the dial is turned. The user can dial the bridge control actuator 907 partially to partially deploy the bridge 109 and can dial the bridge control actuator 907 fully to fully deploy the bridge 109.

The main controller 901 further comprises a bridge lock switch 908 that locks the bridge 109 in a deployed position and prevents it from further deploying or collapsing when it is in a desired position. The bridge lock switch 908 must be unlocked in order to collapse the bridge 109.

The main controller 901 further comprises a cutting wire activation actuator 909 that when actuated, moves the cutting wire 101 (FIG. 1) in a reciprocal motion in the illustrated embodiment. In one embodiment, physically turning the actuator 909 causes the cutting wire 101 to move upwards and downwards in a reciprocating movement about a centimeter in each direction.

In other embodiments, other controllers or forms of control may be used to actuate the cutting wire 101, such as a motor turning a rotating wire.

FIG. 10 illustrates the main catheter 103 of the cutter 100 after it has been positioned in a patient's heart 305 for cutting the leaflets of a bioprosthetic valve 1001. As discussed herein, the main catheter 103 travels to the heart 105 over a central wire 120 in an over-the-wire manner. The nosecone 117 is positioned in the left ventricular cavity 310 of the heart 105 when the cutter is in the desired position to be deployed. The sheath 105 has not ben retracted yet in the illustration of FIG. 10.

FIG. 11 depicts the cutter 100 after it has been advanced into position in the bioprosthetic valve 1001 of FIG. 10 and the sheath has been retracted. The foot 106 is positioned against the leaflet 1002 to be cut. The bridge 109 has not yet been deployed.

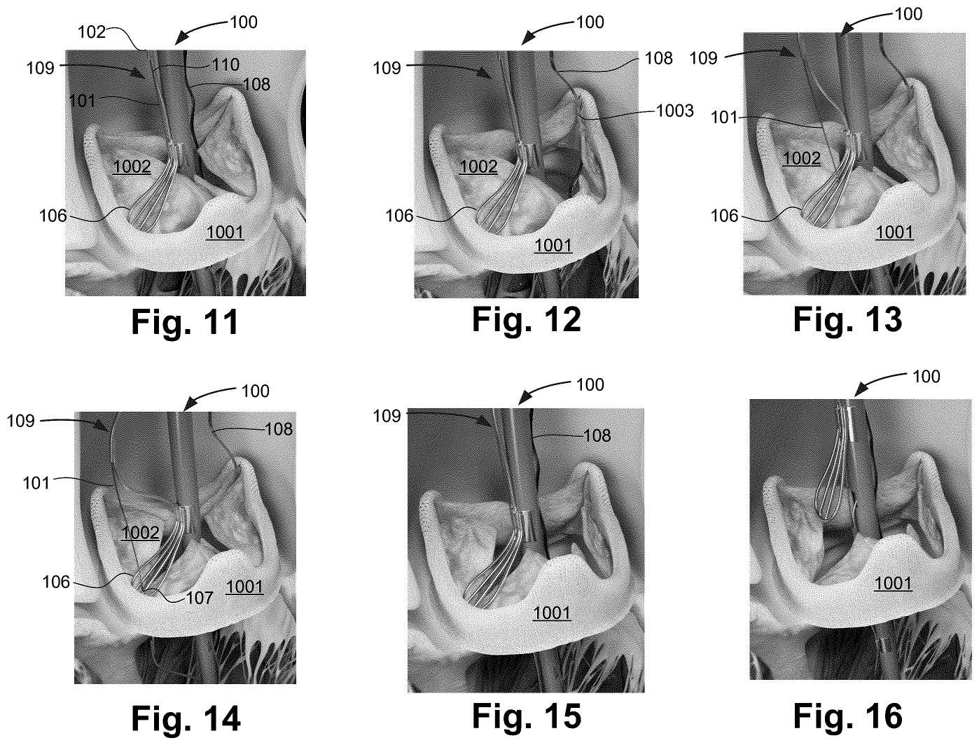

FIG. 12 depicts the cutter 100 of FIG. 11 after the commissure guidewire 108 has been deployed. As was discussed above with respect to FIG. 9, the user deploys the commissure guidewire 108 by actuating the commissure guidewire control actuator 906 (FIG. 9) on the controller 900 (FIG. 9). When deployed, the commissure guidewire 108 rests in the commissure 1003 opposite from the leaflet 1002 to be cut.

FIG. 13 depicts the cutter 100 of FIG. 12 after the bridge 109 has been partially deployed and the leaflet 1002 partially cut. As was discussed above with respect to FIG. 9, the user deploys the bridge 109 by actuating the bridge control actuator 907 (FIG. 9) on the controller 900 (FIG. 9). Before the bridge 109 can deployed, the user must first unlock the bridge lock switch 908 (FIG. 9) as was discussed above with respect to FIG. 9. The user cuts through the leaflet 1002 by actuating the cutting wire actuator 909 (FIG. 9), which moves the cutting wire 101 in a reciprocal fashion in the illustrated embodiment.

FIG. 14 depicts the cutter 100 of FIG. 13 after the bridge 109 has been fully deployed and the leaflet 1002 fully cut. As was discussed above with respect to FIG. 9, the user fully deploys the bridge 109 by further actuating the bridge control actuator 907 (FIG. 9) on the controller 900 (FIG. 9). With the bridge 109 fully deployed, the cutting wire 101 will cut further into the leaflet 1002 when the cutting wire actuator 909 is actuated. However, because the cutting wire 101 is constrained within the inner loop 1005 of the foot 1006, the wire 101 cannot cut too far or cut into the valve 1001.

FIG. 15 depicts the cutter 100 of FIG. 14 after the bridge 109 and commissure guidewire 108 are undeployed following the cutting of the leaflet 1002.

FIG. 16 depicts the cutter 100 of FIG. 15 after the cutter 100 has been partially withdrawn from the valve 1001 to reposition the cutter. The cutter 100 may be rotated by manually rotating the steerable control guide 902 (FIG. 9).

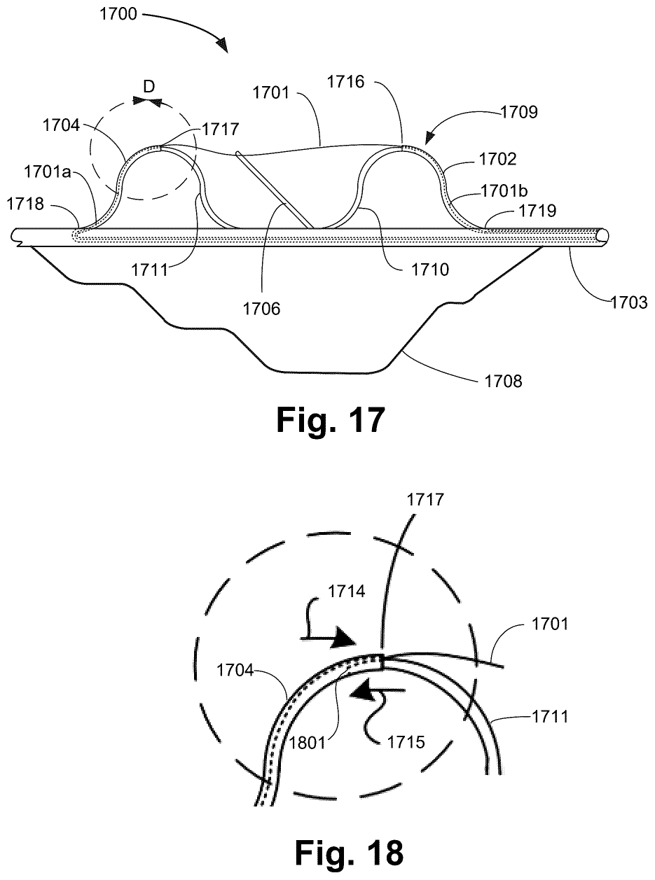

FIG. 17 depicts an alternative embodiment of a bridge 1709 of a cutter 1700 according to the present disclosure. The bridge 1709 comprises a proximal bridge tube 1702 and a distal bridge tube 1704 with a cutting wire 1701 extending between the proximal bridge tube 1702 and the distal bridge tube 1703. The cutting wire 1701 extends from an opening 1716 of the proximal bridge tube 1702 and is fed through the proximal bridge tube 1702 from a controller (not shown) for operation of the cutting wire 1701 as described with reference to FIG. 9 herein. Reference number 1701b depicts the cutting tube within the proximal bridge tube 1702. Similar, the cutting wire 1701 extends from an opening 1717 of the distal bridge tube 1704 and is fed through the distal bridge tube 1704 and returns from the distal bridge tube 1704 to the controller (not shown). Reference number 1701a depicts the cutting tube within the distal bridge tube 1704. Note that the cutting wire 1701 is coextensive with the wires 1701a and 1701b, and the reference numbers 1701a and 1701b are provided for the purpose of illustrating the cutting wire within the distal bridge tube 1704 and proximal bridge tube 1702 respectively.

The bridge 1709 further comprises a proximal bridge support 1710 and a distal bridge support 1711. The proximal bridge support 1710 and the distal bridge support 1711 each comprise hollow tubing (nitinol in one embodiment) with an outer diameter slightly smaller than an inner diameter of the proximal bridge tube 1702 and the distal bridge tube 1704, such that the proximal bridge support 1710 can fit within the proximal bridge tube 1702, and the distal bridge support 1711 can fit within the distal bridge tube 1704, as further discussed below.

FIG. 18 depicts an enlarged view of the distal bridge tube 1704 and the distal bridge support 1711, taken along detail line D of FIG. 17. The end 1801 of the distal bridge support 1711 is partially inside of the opening 1717 of the distal bridge tube 1704 when the bridge 1709 (FIG. 18) is fully deployed. Then, as the bridge 1709 is undeployed, the end 1801 of the distal bridge support 1711 enters the distal bridge tube 1704 further. There is sufficient room within the distal bridge tube 1704 for the cutting wire 1701 and the distal bridge support 1711. The distal bridge tube 1704 receiving the distal bridge support 1711 allows the bridge to collapse such that the sheath (not shown) can slide over the collapsed bridge. In this regard, as the bridge is undeployed, the end 1801 of the distal bridge support 1711 moves in the direction indicated by directional arrow 1715 within the opening 1717 of the distal bridge tube 1704 while the opening 1717 of the distal bridge tube moves in the direction indicated by directional arrow 1714. In this manner, the distal bridge support 1711 is received within the distal bridge tube 1704 as the bridge is undeployed. The proximal bridge tube 1702 and proximal bridge support 1710 are substantially similar to, but mirror images of, the distal bridge tube 1704 and distal bridge support 1711 and operate in a substantially similar manner.

Referring back to FIG. 17, the collapsing of the bridge 1709 to undeploy the cutter 1700 is also aided by the proximal end of the proximal bridge tube 1702 entering the main catheter 1703 via an opening 1719 in the main catheter. The cutting wire 1701b within the proximal bridge tube 1702 are then fed back to the controller.

Similarly, the distal end of the distal bridge tube 1704 enters the main catheter 1703 via an opening 1718 in the main catheter. The cutting wire 1701a within the distal bridge tube 1704 are then fed back to the controller.

The foot 1706 in this embodiment is substantially similar to the foot 106 (FIG. 1) as described herein. The cutting wire 1701 is threaded through the foot 1706 in a substantially similar manner that the cutting wire 101 (FIG. 1) is threaded through the foot 106 (FIG. 1). The commissure guidewire 1708 is substantially similar to the commissure guidewire 108 (FIG. 1) as discussed herein.

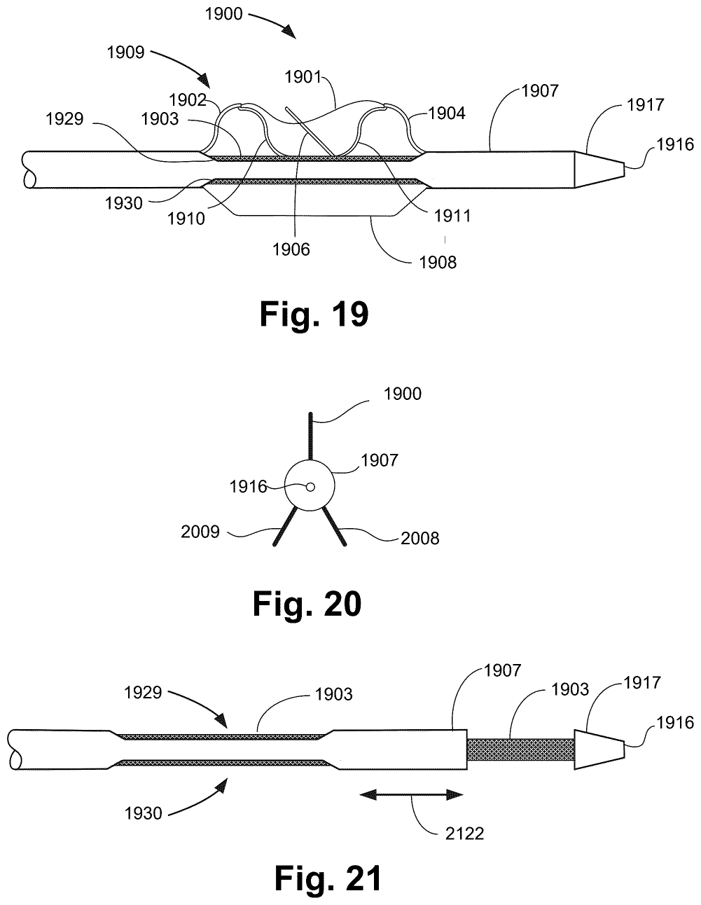

FIG. 19 depicts an alternative embodiment of a mitral valve version of a cutter 1900 according to the present disclosure. The cutter 1900 comprises a bridge 1909 similar to the bridge 109 discussed above with respect to FIG. 1. Because the mitral valve is oriented differently from the other heart valves, a foot 1906 in this embodiment faces away from a nosecone 1917 of the cutter 1900, which is opposite from the direction of the foot 106 (FIG. 1) in the embodiments discussed above. Other than this difference the foot 1906 is substantially similar to those discussed above.

A cutting wire 1901 extends between a proximal bridge tube 1902 and a distal bridge tube 1904 in the same manner as discussed above. The cutting wire 1901 threads through the foot 1906 in the manner as discussed above with respect to the other embodiments disclosed herein. The cutting wire may cut by a reciprocal motion (i.e., sawing), rotational motion, or electrocautery. A proximal bridge support 1910 and a distal bridge support 1911 support the proximal bridge tube 1902 and the distal bridge tube 1904 respectively when the bridge 1909 is in a deployed configuration.

Guidewires 1908 extend from the cutter 1900 when deployed. Although only one guidewire 1908 is illustrated in FIG. 19, there are two actually guidewires 1908 in this embodiment, as further depicted in FIG. 20. The guidewires serve to brace against the posterior mitral valve leaflet (not shown) when the cutter 1900 is in use, serving to stabilize the cutter and provide a forward force in the direction of the anterior mitral valve leaflet (not shown).

The cutter 1900 comprises an outer moveable sheath 1907 that generally covers a main catheter 1903 when the sheath 1907 is fully advanced. A bridge window 1929 in the sheath 1907 over the bridge 1909 allows the bridge 1909 to extend from the cutter 1900 when the bridge 1909 is deployed. Similar, a commissure window 1930 in the sheath 1907 over the commissure guidewires 1908 allows the commissure guidewires 1908 to extend from the cutter 1900 when the commissure guidewires 1908 are deployed. A opening 1916 in the nosecone 1917 receives the wire (not shown) over which the cutter 1900 passes in an over-the-wire fashion.

FIG. 20 depicts an end view of the cutter 1900 of FIG. 19, showing the bridge 1909, which comprises the proximal bridge tube 1902, distal bridge tube 1904, proximal bridge support 1910, distal bridge support 1911, cutting wire 1901, and foot 1906. The two commissure guidewires 2008 and 2009 extend from the cutter 1900 at an opposite side from the bridge 1909, angled radially with respect to each other at an angle of approximately 30 degrees in one embodiment.

FIG. 21 depicts the cutter 1900 of FIG. 19 in an undeployed configuration. The sheath 1907 advances towards and away from the nosecone 1917 in the direction indicated by directional arrow 2122.

In some cases of replacement of a mitral valve (not shown), the new valve will move the anterior leaflet of the mitral valve into a position where it will compromise blood flow out of the heart. This condition is referred to as left ventricular outflow obstruction. By making a cut that is central and longitudinal in the anterior mitral valve leaflet, the left ventricular outflow obstruction can be mitigated or eliminated.

This disclosure may be provided in other specific forms and embodiments without departing from the essential characteristics as described herein. The embodiments described are to be considered in all aspects as illustrative only and not restrictive in any manner.

1. A cardiac cutting device comprising:
a main catheter configured to be threaded into a patient's vasculature, the main catheter comprising a retractable external sheath, the external sheath retracted when the main catheter is at a leaflet to be cut;
a proximal bridge tube and a distal bridge tube retractable within the main catheter and covered by the external sheath when the main catheter is in an undeployed configuration, the proximal bridge tube and a distal bridge tube projecting from the main catheter when the main catheter is in a deployed configuration, the proximal bridge tube and a distal bridge tube projecting radially from the main catheter in a same plane when the main catheter is in the deployed configuration; and
a cutting wire extending between the proximal bridge tube and a distal bridge tube, the cutting wire projecting outwardly from the main catheter when the main catheter is in the deployed configuration, the cutting wire, the proximal bridge tube and the distal bridge tube forming a bridge when the main catheter is in the deployed configuration.

2. The device of claim 1, wherein the cutting wire is operable to cut aortic valve leaflets by moving in a reciprocating motion.

3. The device of claim 1, wherein the cutting wire is operable to cut aortic valve leaflets by moving in a rotating motion.

4. The device of claim 1, further comprising a flexible foot extending from the main catheter when the main catheter is in the deployed configuration, the foot projecting from the main catheter between the proximal bridge tube and a distal bridge tube substantially aligned with the proximal bridge tube and a distal bridge tube, the cutting wire threaded through an inner loop in the foot, the foot retractable within the main catheter when the main catheter is in the undeployed configuration.

5. The device of FIG. 4, wherein the foot angles away from a nosecone at a distal end of the main catheter when the device is configured for cutting mitral valve tissue.

6. The device of FIG. 4, wherein the foot angles towards a nosecone at a distal end of the main catheter when the device is configured for cutting valve leaflets other than mitral valve tissue.

7. The device of claim 1, wherein the cutting wire comprises a diamond wire.

8. The device of claim 1, wherein the proximal bridge tube comprises a proximal bridge tube opening that faces towards the distal end of the catheter and the distal bridge tube comprises a distal bridge tube opening that faces the proximal end of the catheter, the cutting wire extending between the proximal bridge tube opening and the distal bridge tube opening.

9. The device of claim 1, further comprising a proximal bridge support affixed to the proximal bridge tube, the proximal bridge support configured to support the proximal bridge tube when the cutter is in the deployed configuration.

10. The device of claim 9, further comprising a distal bridge support affixed to the distal bridge tube, the distal bridge support configured to support the distal bridge tube when the cutter is in the deployed configuration.

11. The device of claim 1, further comprising a controller for actuating the cutting wire from outside of the patient's body, the cutting wire configured to extend from the main catheter to the controller, the controller comprising a cutting wire activation actuator for reciprocating the cutting wire when the main catheter is in the deployed configuration.

12. The device of claim 11, wherein the controller further comprises a commissure guide control actuator configured to deploy and undeploy the commissure guidewire when the main catheter is in the deployed configuration.

13. The device of claim 12, wherein the controller further comprises a bridge control actuator, the bridge control actuator operable to partially or fully deploy the bridge when the main catheter is in the deployed configuration.

14. A cardiac cutting device comprising:
a main catheter adapted for femoral insertion into a patient's vasculature in an over-the-wire method, the main catheter comprising a retractable external sheath, the external sheath retracted when the main catheter is in a deployed configuration;
a proximal bridge tube and a distal bridge tube extending radially from the main catheter when the main catheter is in the deployed configuration, the proximal bridge tube and a distal bridge tube projecting radially from the main catheter in a same plane;
a cutting wire extending between the proximal bridge tube and a distal bridge tube.

15. The device of claim 14, wherein the proximal bridge tube and a distal bridge tube are retractable within the main catheter and covered by the external sheath when the main catheter is in an undeployed configuration.

16. The device of claim 14, wherein the cutting wire is operable to cut valve leaflets by moving in a reciprocating motion.

17. The device of claim 14, further comprising a flexible foot extending from the main catheter when the main catheter is in the deployed configuration, the foot projecting from the main catheter between the proximal bridge tube and a distal bridge tube substantially aligned with the proximal bridge tube and a distal bridge tube, the cutting wire threaded through an inner loop in the foot, the foot retractable within the main catheter when the main catheter is in the undeployed configuration.

18. The device of claim 14, further comprising a proximal bridge support affixed to the proximal bridge tube, the proximal bridge support configured to support the proximal bridge tube when the cutter is in the deployed configuration.

19. The device of claim 18, further comprising a distal bridge support affixed to the distal bridge tube, the distal bridge support configured to support the distal bridge tube when the cutter is in the deployed configuration.

20. The device of claim 14, further comprising a controller for actuating the cutting wire from outside of the patient's body, the cutting wire configured to extend from the main catheter to the controller, the controller comprising a cutting wire activation actuator for reciprocating the cutting wire when the main catheter is in the deployed configuration.
